(19) United States
(12) Patent Application Publication
(10) Pub. No.: US 2026/0250218 A1
(43) Pub. Date: Aug. 27, 2026

(54) RECOVERY OF STYRENE MONOMER FROM POLYSTYRENE

(71) Applicant: T.EN Process Technology, Inc., Houston, TX (US)

(72) Inventors: Douglas Stuart HUBBELL, Sudbury, MA (US); Slawomir A. OLESKY, Billerica, MA (US)

(21) Appl. No.: 18/993,883

(22) PCT Filed: Jun. 30, 2023
(86) PCT No.: PCT/US2023/026702

§ 371 Date: Jan. 13, 2025

Related U.S. Application Data
(60) Provisional application No. 63/388,856, filed on Jul. 13, 2022.

(51) Int. Cl. C07C 4/22; B01D 3/14; B01J 6/00; C07C 7/00

(52) U.S. Cl. CPC C07C 4/22; B01D 3/143; B01J 6/008; C07C 7/005

(57) ABSTRACT

In a process for recovering styrene monomer from polystyrene, a feed comprising benzene, toluene, ethylbenzene, styrene monomer and C₉₊ aromatics produced by depolymerizing polystyrene is supplied to a first fractionation column and divided into a first bottoms fraction containing part of the C₉₊ aromatics in the feed and a first overhead fraction composed of the remainder of the feed. The first overhead fraction is supplied to a second fractionation column and divided into a second overhead fraction rich in benzene, toluene, and ethylbenzene compared to the first overhead fraction and a second bottoms fraction rich in styrene monomer and C₉₊ aromatics compared to the first overhead fraction. The second bottoms fraction is supplied to a third fractionation column and divided into a third overhead fraction rich in styrene monomer compared to the second bottoms fraction and a third bottoms fraction rich in C₉₊ aromatics compared to the second bottoms fraction.

### FIELD

This application relates to a process for the recovery of styrene monomer from the reaction products of the depolymerization of polystyrene.

### BACKGROUND

Environmental pollution, resource scarcity and climate change have all been instrumental in driving the search for a cyclical, rather than a linear, economy. For example, intensive efforts have been underway for thirty years to develop processes for the recovery of the raw materials of plastic waste. While these efforts have not yet resulted in large-scale applications, the growing problem of plastic waste has led to a burgeoning interest in chemical recycling.

Not all thermoplastic polymers are equally suited for chemical recycling. For example, the thermal decomposition of polyolefins results in mixtures of waxes, light oils and gases, while the degradation of polyethylene terephthalate (PET) results in organic acids, mainly benzoic acid and terephthalic acid, which are corrosive and may cause reactor clogging. On the other hand, polystyrene can be depolymerized by pyrolysis or other means to monomeric styrene, making it an excellent candidate for chemical recycling. However, the product mixture of such depolymerization processes needs to be purified in order to use the product styrene as a raw material for new polymer products.

For example, the pyrolysis of polystyrene not only produces styrene monomer but also results in the formation of a wide variety of heavier, nonpolymeric aromatic compounds, such as diphenylpropane and naphthalene. The pyrolysis is also typically incomplete in that there still are styrene dimers, trimers and other oligomers in the pyrolysis product that did not fully depolymerize. Lighter by-products, e.g., benzene, toluene, ethylbenzene (EB), cumene and alpha-methylstyrene, are also typically formed. Such by-products can have a serious detrimental influence on the utility of the styrene monomer produced. For example, styrene oligomers are detrimental to the use of the monomer in repolymerization processes since, even in small quantities, these oligomers can change important properties of the polymer. Similarly, other aromatic byproducts can act as chain transfer agents in radical-based polymerization processes, lowering the average molecular weight of the polymers produced and contributing to polymers with a lower glass transition temperature.

In addition, polystyrene products often also contain one or several additives, like brominated flame retardants, which need to be removed in order to generate styrene monomer that is suitable to produce many products including food packaging. Bromine from flame retardants may also lead to acidic or toxic gases, such as hydrobromic acid (HBr), during thermal decomposition of waste polystyrene and can also form brominated organic compounds.

Thus the commercial viability of styrene monomer produced from the depolymerization of polystyrene depends on the development of an efficient and economic process and apparatus for separating the styrene monomer from the byproducts of the depolymerization process.

One proposed process is described in International Patent Publication No. WO 2020/144165, in which the polystyrene pyrolysis product is condensed and the condensate is supplied to a first fractionation column, where a light fraction containing benzene, toluene and some ethylbenzene is removed as distillate, an intermediate fraction containing most of the styrene monomer and some ethylbenzene is removed as a side cut, and a heavies fraction containing styrene oligomers is removed as bottoms. The intermediate fraction is then sent to a second fractionation column, where most of the remaining ethylbenzene is removed as distillate and a fraction rich in styrene monomer is removed as bottoms. The bottoms fraction from the second fractionation column is then sent to a third fractionation column, where most of the remaining substances with higher boiling points than styrene monomer are removed.

However, the process described in WO 2020/144165 suffers from a number of disadvantages. First, the first fractionation column is required to have a large number of theoretical stages with reflux and a side draw to achieve the desired separations, which makes this column system expensive to install and operate at a bottoms temperature at or above 200° C. The latter poses a particular problem since the condensate from polystyrene pyrolysis has been found to contain significantly higher amounts of known cross-linking compounds, such as divinylbenzene (DVB), than in the reaction product from the dehydrogenation of ethylbenzene, which is the conventional process for making styrene. When present in concentrations much above 100 ppm relative to styrene at typical styrene distillation temperatures, DVB can lead to formation of cross-linked, insoluble polystyrene which may foul the internals of fractionation columns and their reboilers. This type of fouling can require lengthy shutdowns to clean or replace column internals and other equipment.

There is therefore significant interest in developing improved processes and apparatus for the recovery of styrene monomer from the reaction products of the depolymerization of polystyrene.

### SUMMARY

According to the present application, it has now been found that, by feeding the condensed product from a polystyrene depolymerization process to a small stripping column provided upstream of the other purification columns, it is possible to remove some of the C₉ by-products and virtually all of the C₁₀₊ heavies, including a significant portion of the DVB, before any separation of the lighter components from the desired styrene monomer. Since this stripping column only removes the heavies, it requires fewer theoretical stages than if a distillate product of by-products lighter than styrene is also produced in that first column.

In one aspect, the present application provides a process for the recovery of styrene monomer from the reaction products of the depolymerization of polystyrene, the process comprising:
(a) supplying a feed stream comprising benzene, toluene, ethylbenzene, styrene monomer and C₉₊ aromatic compounds produced by the depolymerization of polystyrene to a first fractionation column to divide the feed stream into a first bottoms fraction containing part of the C₉₊ aromatic compounds in the feed stream and a first overhead fraction, which is vapor, composed of the remainder of the feed stream;
(b) supplying the first overhead fraction to a second fractionation column to divide the first overhead fraction into a second overhead fraction rich in benzene, toluene and ethylbenzene compared to the first overhead fraction and a second bottoms fraction rich in styrene monomer and C₉₊ aromatic compounds compared to the first overhead fraction; and
(c) supplying the second bottoms fraction to a third fractionation column to divide the second bottoms fraction into a third overhead fraction rich in styrene monomer compared to the second bottoms fraction and a third bottoms fraction rich in C₉₊ aromatic compounds compared to the second bottoms fraction.

In a further aspect, the present application provides apparatus for the recovery of styrene monomer from the reaction products of the depolymerization of polystyrene, the apparatus comprising:
(a) a first fractionation column that receives a depolymerization oil comprising part of the reaction products of the depolymerization of polystyrene, the first distillation column being operated to divide the depolymerization oil into a first bottoms fraction containing part of the C₉₊ aromatic compounds in the depolymerization oil and a first overhead fraction, which is vapor, composed of the remainder of the depolymerization oil;
(b) a second fractionation column connected to the first fractionation column to receive the first overhead fraction and operated to divide the first overhead fraction into a second overhead fraction rich in benzene, toluene, and ethylbenzene compared to the first overhead fraction and a second bottoms fraction rich in styrene monomer and C₉₊ aromatic compounds compared to the first overhead fraction; and
(c) a third fractionation column connected to the second fractionation column to receive the second bottoms fraction and operated to divide the second bottoms fraction into a third overhead fraction rich in styrene monomer compared to the second bottoms fraction and a third bottoms fraction rich in C₉₊ aromatic compounds compared to the second bottoms fraction.

### BRIEF DESCRIPTION OF THE DRAWINGS

FIG. 1 is a simplified flow diagram of a process and apparatus according to one embodiment of the present application for the recovery of styrene monomer from the reaction products of the depolymerization of polystyrene.

FIG. 2 is a flow diagram similar to FIG. 1 of another embodiment of the present application.

### DETAILED DESCRIPTION OF THE EMBODIMENTS

As used herein, the term “Cₙ” compound (hydrocarbon) wherein n is a positive integer, e.g., 1, 2, 3, 4, 5, etc., means a compound having “n” number of carbon atom(s) per molecule. The term “Cₙ₊” compound wherein “n” is a positive integer, e.g., 1, 2, 3, 4, 5, etc., means a compound having at least “n” number of carbon atom(s) per molecule. The term “Cₙ₋” compound wherein n is a positive integer, e.g., 1, 2, 3, 4, 5, etc., as used herein, means a compound having no more than n number of carbon atom(s) per molecule.

Described herein are a process and apparatus for the recovery of styrene monomer from the reaction products of the depolymerization of polystyrene. In one embodiment, the present process employs a feed comprising benzene, toluene, ethylbenzene, styrene monomer, and C₉₊ aromatic compounds, including styrene oligomers, in which the feed is produced by pyrolyzing polystyrene to produce a pyrolysis gas and then condensing most of the aromatic hydrocarbons so as to separate them from most of the C₅₋ light products. The resultant liquid feed, which is also referred to herein as depolymerization oil, is then supplied to a first fractionation column which is operated to divide the feed into a first bottoms fraction containing part of the C₉₊ aromatic compounds in the feed and a first overhead fraction, which is a vapor, composed of the remainder of the feed, mostly benzene, toluene, ethylbenzene, and styrene monomer. No side cut containing styrene monomer and ethylbenzene is removed from the first fractionation column in the present process. The first overhead fraction from the first fractionation column is then supplied to a second fractionation column which is operated to divide the first overhead fraction into a second overhead fraction rich in benzene, toluene, ethylbenzene compared to the first overhead fraction and a second bottoms fraction rich in styrene monomer and C₉₊ aromatic compounds compared to the first overhead fraction. The second bottoms fraction from the second fractionation column is then supplied to a third fractionation column which is operated to divide the second bottoms fraction into a third overhead fraction rich in styrene monomer compared to the second bottoms fraction and a third bottoms fraction rich in C₉₊ aromatic compounds compared to the second bottoms fraction.

The depolymerization oil employed as feed in the present process may be produced in situ or may be transported to the site of the separation system described above from one or more separate locations. One type of polystyrene depolymerization process is pyrolysis. In the pyrolysis reactor, polystyrene is heated in the absence of oxygen to a temperature sufficient to depolymerize the polystyrene into styrene monomer. The resultant pyrolysis gas generally contains in excess of 60 wt. % of styrene monomer together with smaller amounts of benzene, toluene, other C₈ aromatic compounds, such as phenylacetylene and xylenes, and C₉₊ aromatic compounds, including styrene oligomers, as well as various C₅₋ light by-products. The pyrolysis gas is fed to a condenser where the gas is typically cooled to near ambient temperature to condense out the benzene and heavier components, leaving the C₅₋ light by-products in the vapor phase so that they can be separated from the aromatic hydrocarbons and potentially used as fuel gas.

A typical analysis of the condensed depolymerization oil produced from pyrolysis of polystyrene is shown in Table 1 which also lists, for comparison purposes, the typical commercial composition of the crude styrene liquid resulting from the dehydrogenation of ethylbenzene, which is the most common industrial process for making styrene.

TABLE 1

| | | Crude styrene |
|---|---|---|
| | Depolymerization | liquid from |
| | oil | EB dehydrogenation |
| | (wt. %) | (wt. %) |
| C₅− lights | 0.1 | 0.03 |
| Benzene | 0.5 | 1 |
| Toluene | 5 | 1 |
| Ethylbenzene (EB) | 4 | 37 |
| Xylenes | 0.03 | 0.001 |
| Styrene | 75 | 60 |
| Phenylacetylene | 0.001 | 0.01 |
| Cumene | 0.03 | 0.007 |
| α-Methylstyrene | 5 | 0.06 |
| Divinylbenzene | 0.03 | 0.006 |
| Other C₉-C₁₀ | 1 | 0.015 |
| C₁₁+ components | 9 | 0.5 |

It will be seen from Table 1 that, although the composition of depolymerization oil is similar to that of the crude styrene liquid from EB dehydrogenation, the former contains much less EB, whereas in the case of the latter, it is necessary to separate the benzene-toluene fraction from EB since the latter is recycled back to the dehydrogenation section. This separation is not needed in the case of depolymerization oil, since the amount of EB is relatively small (typically less than 10 wt. %), and the entire benzene/toluene/EB fraction can be sent for processing to an aromatics plant. It will also be seen that the concentrations of cumene and α-methylstyrene are much higher in the polystyrene depolymerization oil and these two compounds are potentially major impurities in the purified styrene product. Cumene is especially difficult to fractionate from styrene.

It is also important to note that the concentration of divinylbenzene (DVB) is several times higher in the depolymerization oil. DVB is a known crosslinking agent, and when it is present in concentrations much above 100 ppm relative to styrene it can lead to formation of cross-linked, insoluble polystyrene which may foul the internals of the distillation columns and their reboilers. This type of fouling can require lengthy shutdowns to clean or replace column internals and equipment. It can be anticipated that the fouling potential is higher when recovering styrene from depolymerization oil than in the case of recovering styrene from crude styrene from EB dehydrogenation because the DVB concentration is much higher.

To separate the components of the depolymerization oil, the latter is first mixed with a polymerization inhibitor, such as a mono- or dinitro-aromatic compound, and then the mixture is fed, without initial fractionation, to a first fractionation column. Alternatively, the polymerization inhibitor may be fed separately to the first fractionation column. In embodiments, the first fractionation column is a reboiled stripper in that the column includes a reboiler for the liquid column bottoms but not a condenser for the vapor overhead. The first fractionation column is operated to divide the depolymerization oil feed into a first bottoms fraction containing part of the C₉₊ aromatic compounds, and preferably virtually all of the C₁₀₊ aromatic compounds, in the feed and a first overhead vapor fraction, consisting of the remainder of the feed. In embodiments, the first stripping column separates a sufficient portion of the divinylbenzene in the depolymerization oil feed from most of the styrene so that crosslinking of polymer is not a significant problem in the downstream columns. A majority of the divinylbenzene leaves as part of the first bottoms fraction, and most of the styrene leaves in the overhead vapor. The entire overhead fraction from the first stripping column is forwarded as a vapor to the second fractionation column, whereas the first bottoms fraction is a net product of the system.

The first stripping column typically has between 1 and 15 theoretical stages, such as from 5 to 10 theoretical stages, with the depolymerization oil feed being introduced into the first stripping column at or close to the uppermost stage. Although the internals of the first stripping column can include fractionation packing, it is preferable to employ distillation trays since these are less expensive and easier to clean of insoluble polymer build-up than packing. In embodiments, the first stripping column is operated at a bottom temperature from 90 to 155° C., such as from 100 to 150° C., and a top or head temperature from 55 to 100° C., such as 65 to 90° C., and at a bottom pressure from 40 to 300 mmHg, such as 70 to 200 mmHg, and a top or head pressure from 40 to 180 mmHg, such as 50 to 120 mmHg. The first stripping column includes a reboiler to redistill part of the first bottoms fraction. However, the first stripping column docs not include a side cut for separating the EB and styrene monomer from the lighter components and preferably does not have a condenser and associated pump for refluxing part of the overhead fraction.

The first overhead fraction contains virtually all the benzene and toluene, and most of the C₈ aromatic components, including the styrene monomer, in the depolymerization oil feed and is fed to the second fractionation column. Polymerization inhibitor can also be added to the second column. The second fractionation column may include distillation trays but typically is a packed column. In embodiments, the second fractionation column has at least 20 theoretical stages, such as from 20 to 120 theoretical stages, more preferably from 40 to 100 theoretical stages. Because the first stripping column does not include reflux from the condensed first column overhead, essentially all the heat put into the first column reboiler is in the vapor sent to the second fractionation column leading to an overall improvement in energy efficiency relative to a column system with a condenser and reflux. In embodiments, the second fractionation column is a complete distillation system with a reboiler for the liquid column bottoms and a condenser for the vapor column overhead. Typically, the second fractionation column is operated at a bottom temperature from 60 to 120° C., such as 70 to 105° C., and a top or head temperature from 35 to 100° C., such as 40 to 70° C., and at a bottom pressure from 45 to 370 mmHg, such as from 65 to 210 mmHg, and a top or head pressure from 30 to 290 mmHg, such as from 40 to 145 mmHg. Under these conditions, the second fractionation column is effective to divide the first overhead fraction into a second overhead fraction rich in benzene, toluene, and ethylbenzene compared to the first overhead fraction, and a second bottoms fraction rich in styrene monomer and C₉₊ aromatic compounds compared to the first overhead fraction.

The gaseous second overhead fraction exiting the second fractionation column is passed through a condenser where the majority of the second overhead fraction is condensed. Part of the resultant condensate is then returned to the second fractionation column as reflux, while some or all of the remainder is recovered as a byproduct (distillate) stream, which can be a feed to an aromatics plant, with the ratio of reflux to distillate typically lying between 15 to 30. In some embodiments, part of the condensate is fed back to the top of the first stripping column as a reflux stream, with the ratio of condensate fed back to the first column to the distillate stream typically lying between 0.2 and 0.4. Any water in the depolymerization oil will mostly condense in the second fractionation column condenser and form a separate liquid phase. Optionally, this liquid water can be separated off from the liquid hydrocarbon.

The depolymerization oil can contain hydrochloric acid (HCl), hydrobromic acid (HBr), and other halogenated compounds. If the aqueous phase is separated as a separate stream as described above, then these halogenated compounds will be in this aqueous phase to some extent. To enhance this extraction of halogenated compounds and other impurities from the second column hydrocarbon distillate, water can be added to the overhead system of the second fractionation column. To further enhance extraction of certain impurities, the second column distillate can be fed to an extraction column to countercurrently wash the hydrocarbon with water in one or more equilibrium stages.

The net liquid bottoms from the second fractionation column, composed mainly of styrene monomer and C₉₊ aromatic compounds, is supplied to the third fractionation column, optionally together with additional polymerization inhibitor. To maximize product separation in the second fractionation column, part of the second column bottoms stream is fed to a reboiler, heated and then returned to the bottom of the second fractionation column.

The third fractionation column may include distillation trays but typically is a packed column. Generally, the third fractionation column has from 10 to 120 theoretical stages, for example from 30 to 100 theoretical stages. In embodiments, the third fractionation column is a complete distillation system with a reboiler for the liquid column bottoms and a condenser for the vapor column overhead. Typically, the third fractionation column is operated at a bottom temperature from 55 to 130° C., such as from 75 to 115° C., and a top or head temperature from 35 to 100° C., such as from 40 to 80° C., and at a bottom pressure from 15 to 230 mmHg, such as from 35 to 160 mmHg, and a top or head pressure from 10 to 150 mmHg, such as from 15 to 90 mmHg. Under these conditions, the third fractionation column operates to divide the second bottoms fraction into a third overhead fraction rich in styrene monomer compared to the second bottoms fraction and a third bottoms fraction rich in C₉₊ aromatic compounds compared to the second bottoms fraction.

The gaseous third overhead fraction exiting the third fractionation column is passed through a condenser where the majority of the third overhead fraction is typically condensed. Part of the resultant condensate may then be returned to the third fractionation column as reflux, with the ratio of reflux to distillate typically lying between 2.5 to 6, while the remainder (distillate) is recovered as the desired styrene monomer product, typically at a purity of at least 99.0% by weight, preferably at least 99.8% by weight. The resultant styrene monomer product typically contains no more than 20 ppmw of halogenated compounds, preferably less than 10 ppmw of halogenated compounds, and more preferably less than 1 ppmw halogenated compounds or below the detection level of typically available testing methods for halogenated compounds in aromatic hydrocarbons. The third bottoms fraction may be combined with the first bottoms fraction and purged from the system.

Halogenated compounds may appear in the styrene monomer product. To extract these halogenated compounds from the third column hydrocarbon distillate, water can be added to the overhead system of the third fractionation column. To further enhance extraction of certain impurities, the third column distillate can be fed to an extraction column to countercurrently wash the hydrocarbon with water in one or more equilibrium stages.

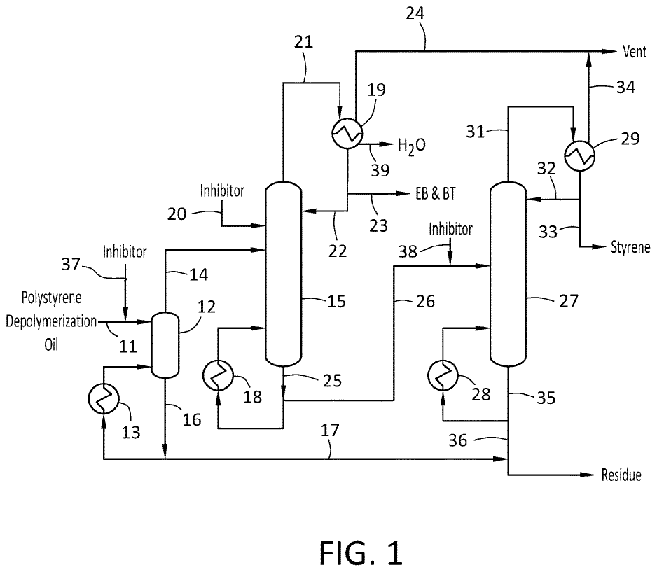

One embodiment of the present process is shown in FIG. 1, in which a polystyrene depolymerization oil is mixed with a polymerization inhibitor from line 37 and fed via line 11 to the top of a first fractionation column 12 equipped with distillation trays and a reboiler 13. The first fractionation column 12 is constructed and operated as described above to divide the oil/inhibitor mix into two fractions; a first overhead vapor fraction rich in the C₈ and lighter components of the feed and a first bottoms fraction rich in the C₉₊ components of the feed. The first overhead fraction exits the top of the column 12 through line 14 and is fed as a vapor to a second fractionation column 15, while the liquid from the base of the column 12 flows through line 16 where it is partitioned between the reboiler 13 and a first column bottoms fraction in line 17, which is a byproduct stream.

The second fractionation column 15 contains distillation packing and is equipped with a reboiler 18 and a condenser 19. Additional polymerization inhibitor can be supplied to the second fractionation column 15 via line 20. The column 15 is constructed and operated as described above to divide the first overhead fraction into a second overhead fraction rich in components boiling at lower temperatures than styrene monomer and a second bottoms fraction rich in styrene monomer and higher boiling components. The second column overhead vapor exits the top of the column 15 and is fed via line 21 to the condenser 19, where the benzene, toluene and ethylbenzene components are mostly condensed and partitioned between a reflux line 22 for recycle to the column 15 and a product recovery line 23. Some C₈₋ components in the second overhead fraction leave the condenser 19 as vapor via vent line 24. Optionally, any condensed water can be separated from the hydrocarbon liquid phase and taken off in line 39. The second fractionation column bottoms exits the base of the column 15 through line 25 where part of the second column bottoms is recycled through the reboiler 18 to the column 15 and the remainder is the second column bottoms fraction which is fed via line 26 to a third distillation column 27. Additional polymerization inhibitor from line 38 can be mixed with the second bottoms fraction in line 26 before it enters the third fractionation column 27.

The third fractionation column 27 contains distillation packing and is equipped with a reboiler 28 and a condenser 29. The column 27 is constructed and operated as described above to divide the second bottoms fraction into a third overhead fraction rich in styrene monomer and components boiling at lower temperatures than styrene monomer and a third bottoms fraction rich components boiling at higher temperatures than styrene monomer. The third column overhead vapor exits the top of the column 27 and is fed via line 31 to the condenser 29, where most of the styrene monomer is condensed and partitioned between a reflux line 32 for recycle to the column 27 and the third column overhead fraction which flows through product recovery line 33. Any uncondensed vapor from condenser 29 exits through line 34. The third column bottoms flow exits the base of the column 27 through line 35 where part is recycled through the reboiler 28 to the column 27 and the remainder is the third column bottoms fraction that flows through line 36 and is a process byproduct. The column system vents from the second and third columns flowing through lines 24 and 34 flow either individually to separate vacuum systems (not shown) or can be combined and flow to one vacuum system.

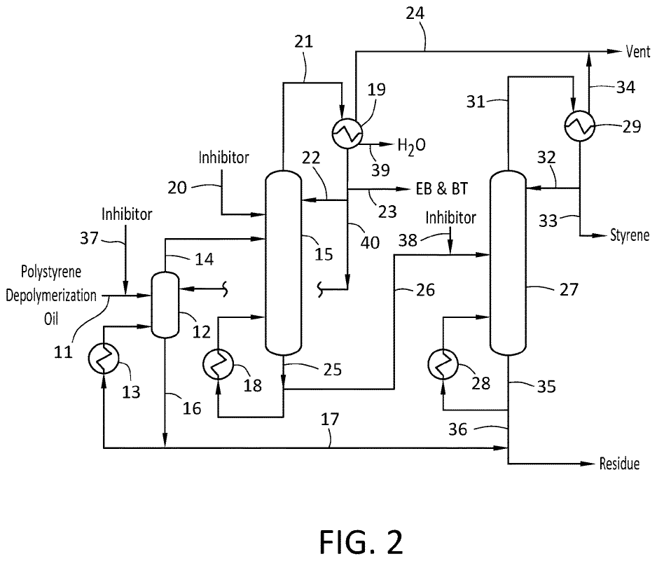

Another embodiment of the present invention is shown in FIG. 2, in which the process shown in FIG. 1 has been modified to include a stream of condensed overhead from the second fractionation column 15 being fed through line 40 to the top stage of the first fractionation column 12 and the depolymerization oil stream in line 11 being fed to a lower stage in the first fractionation column 12, preferably the second from the top stage. This added stream reduces the concentration of divinylbenzene in the feed to the second fractionation column 15 in line 14 and/or reduces the number of theoretical stages needed in the first fractionation column 12 to achieve the desired separation. Recycling a distillate from one fractionation column to a previous column would be expected by one of ordinary skill in the art to result in a significant inefficiency. However, in the present case, the recycle flow required to achieve a substantial reduction in the divinylbenzene concentration in line 14 is minor, and the impact on the reboiler duties in the process is surprisingly small.

The invention will now be more particularly described with reference to the following non-limiting Examples.

### Example 1

Pyrolysis oil containing by weight 78% styrene, 0.6% benzene and lighter compounds, 6.4% toluene, 4.7% ethylbenzene, 0.15% cumene, 520 ppm divinylbenzene, 10% other C₉₊ aromatic compounds, and 20 ppm bromides was fed to the first column 12 of a laboratory distillation system as shown in FIG. 1. The first column 12 was a trayed column with 10 theoretical stages and was maintained with a temperature of 148.0° C. and pressure of 88 mmHg in the bottom and a temperature of 74.2° C. and pressure of 73 mmHg in the overhead with no reflux. The pyrolysis oil was fed to the top tray of the column 12 at an average rate of 473 g/hr. The bottoms stream from column 12 was collected at an average rate of 45 g/hr and was the only stream to contain detectable bromides; its composition by weight was 0.28% styrene, less than 0.06% ethylbenzene and lighter compounds, 10 ppm cumene, 0.39% divinylbenzene, and the balance other C₉₊ aromatic compounds. The styrene-rich overhead from the column 12 flowed as vapor to the second column 15 at an average rate of 428 g/hr. The second column 15 had 48 theoretical stages with the bottom maintained at 101.8° C. and 181 mmHg and the overhead maintained at 56.7° C. and 69 mmHg. The external reflux ratio was maintained within a range of 18 to 22. The liquid distillate of the second column 15 was collected at an average rate of 52 g/hr and contained by weight 11% styrene, 33% ethylbenzene, 52% toluene, and 4% lighter compounds (mostly benzene). The bottoms of the second column 15 was fed at an average rate of 376 g/hr to the third column 27 with 32 theoretical stages. The third column 27 was maintained at 107.8° C. and 125 mmHg in the bottom and 71.0° C. and 53 mmHg in the overhead. The external reflux ratio of this third column was maintained within a range of 3 to 4. The styrene-rich distillate from the third column was collected at an average rate of 302 g/hr and was by weight 99.9% styrene with the largest impurities being ethylbenzene at 230 ppm and cumene at 340 ppm. An inductively coupled plasma test detected no bromine in the third column distillate, indicating a concentration below the detection limit of 1.0 ppmw. Polymerization inhibitor chemicals were injected to the first column 12 such that the column feed was 1200 ppm by weight inhibitor.

### Example 2

Simulations of the FIG. 1 and FIG. 2 embodiments were conducted using commercial process modeling software. The composition of pyrolysis oil from Table 1, was fed to a simulated column 12 of a distillation system at a rate of 1000 kg/hr. In one simulation, the column 12 was modeled as per the embodiment shown in FIG. 1, with the feed fed to the top stage and no reflux supplied to this first column 12. In the second simulation, column 12 was modeled with the feed fed to the second from the top stage and a reflux stream 40 returned to the top stage as in the embodiment shown in FIG. 2 at a rate of 50 kg/hr. The first column 12 was modeled with 9 theoretical stages with a temperature of 108° C. and a pressure of 88.4 mmHg in the bottom and a pressure of 73 mmHg and a temperature of around 73° C. in the overhead. The simulated column 12 was controlled such that the bottoms stream contained no more than 10% styrene by weight. The bottoms rate was calculated to be about 137 kg/hr. The overhead from column 12 flowed as vapor to a second simulated column 15; the overhead rate was different between the two embodiments, see Table 2. The second column 15 was simulated with 51 theoretical stages with a temperature of 99° C. and a pressure of 181 mmHg in the bottom and a temperature of 59° C. and a pressure of 69 mmHg in the overhead. The column 15 was controlled such that the distillate composition was by weight 10% styrene; the remainder of the distillate was calculated to be 1.4% benzene and lighter components, 32.2% toluene, and 56.1% ethylbenzene. The distillate rate was calculated at 22.5 kg/hr; for the simulation of the FIG. 2 embodiment, this distillate rate is net of the reflux stream 40. The bottoms of column 15 was calculated with a rate of 758 kg/hr and was fed to a third column 27. The third column 27 was modeled with 46 theoretical stages with a temperature of 66° C. and a pressure of 53 mmHg in the overhead and a temperature of 104° C. and 125 mmHg in the bottom. The simulated column 27 was controlled such that the distillate was by weight 99.9% styrene; the remaining distillate composition by weight was 209 ppm ethylbenzene, 159 ppm cumene, and the balance heavier compounds. The distillate rate was calculated to be 721 kg/hr. Other quantities and parameters differed between the two simulations, which are compared in Table 2 below.

TABLE 2

| | Simulation | Simulation |
|---|---|---|
| | of FIG. 1 | of FIG. 2 |
| | Embodiment | Embodiment |
| Reflux to Column 12, | 0 | 50 |
| kg/hr | | |
| Column 12 bottoms | 0.195 | 0.201 |
| DVB composition, wt % | | |
| Column 12 reboiler | 108 | 113 |
| duty, Mcal/hr | | |
| Column 12 distillate | 865 | 916 |
| rate, kg/hr | | |
| Column 12 distillate | 37 | 26 |
| DVB composition, ppm | | |
| Column 15 reboiler | 388 | 425 |
| duty, Mcal/hr | | |

While the present invention has been described and illustrated by reference to particular embodiments, those of ordinary skill in the art will appreciate that the invention lends itself to variations not necessarily illustrated herein. For this reason, then, reference should be made solely to the appended claims for purpose of determining the true scope of the present invention.

1. A process for the recovery of styrene monomer from the reaction products of the depolymerization of polystyrene, the process comprising:
(a) supplying a feed stream comprising benzene, toluene, ethylbenzene, styrene monomer and C₉₊ aromatic compounds produced by depolymerizing polystyrene to a first fractionation column to divide the feed stream into a first bottoms fraction containing part of the C₉₊ aromatic compounds in the feed stream and a first overhead fraction, which is vapor, composed of the remainder of the feed stream;
(b) supplying the first overhead fraction to a second fractionation column to divide the first overhead fraction into a second overhead fraction rich in benzene, toluene, and ethylbenzene compared to the first overhead fraction and a second bottoms fraction rich in styrene monomer and C₉₊ aromatic compounds compared to the first overhead fraction; and
(c) supplying the second bottoms fraction to a third fractionation column to divide the second bottoms fraction into a third overhead fraction rich in styrene monomer compared to the second bottoms fraction and a third bottoms fraction rich in C₉₊ aromatic compounds compared to the second bottoms fraction.

2. The process of claim 1, wherein the first fractionation column has no more than 15_theoretical stages.

3. The separation system of claim 2, wherein the first fractionation column has from 5 to 10 theoretical stages.

4. The process of claim 1, m wherein the first fractionation column is operated at a bottom temperature from 90 to 155° C. and a top temperature from 55 to 100° C.

5. The process of claim 1, wherein the first fractionation column is a trayed column.

6. The process of claim 1, wherein each of the second and third fractionation columns is a packed column.

7. The process of claim 1, wherein the second fractionation column has at least 20 theoretical stages.

8. The process of claim 1, wherein one or more polymerization inhibitors are fed to the respective fractionation column during one or more of the supplying steps (a) and (b).

9. The process of claim 1, wherein part of the second overhead fraction is condensed and used as reflux for the first fractionation column.

10. The process of claim 1, wherein water is used to extract halogenated compounds and other impurities from at least part of the second overhead fraction and/or at least part of the third overhead fraction.

11. The process of claim 1, wherein the styrene monomer product contains less than 10 ppmw halogenated compounds.

12. An apparatus for the recovery of styrene monomer from the reaction products of the depolymerization of polystyrene, the apparatus comprising:
(a) a first fractionation column that receives a depolymerization oil comprising part of the reaction products of the depolymerization of polystyrene, the first distillation column being operated to divide the depolymerization oil into a first bottoms fraction containing part of the C₉₊ aromatic compounds in the depolymerization oil and a first overhead fraction, which is vapor, composed of the remainder of the depolymerization oil;
(b) a second fractionation column connected to the first fractionation column to receive the first overhead fraction and operated to divide the first overhead fraction into a second overhead fraction rich in benzene, toluene, and ethylbenzene compared to the first overhead fraction and a second bottoms fraction rich in styrene monomer and C₉₊ aromatic compounds compared to the first overhead fraction; and
(c) a third fractionation column connected to the second fractionation column to receive the second bottoms fraction and operated to divide the second bottoms fraction into a third overhead fraction rich in styrene monomer compared to the second bottoms fraction and a third bottoms fraction rich in C₉₊ aromatic compounds compared to the second bottoms fraction.

13. The apparatus of claim 12, wherein the first fractionation column has less than 15_theoretical stages.

14. The apparatus of claim 13, wherein the first fractionation column has 5 to 10 theoretical stages.

15. The apparatus of claim 12, wherein the first fractionation column is operated at a bottom temperature from 90 to 155° C. and a top temperature from 55 to 100° C.

16. The apparatus of claim 12, wherein the first fractionation column is a trayed column.

17. The apparatus of claim 12, wherein each of the second and third fractionation columns is a packed column.

18. The apparatus of claim 12, wherein the second fractionation column has at least 20 theoretical stages.

19. A system for the recovery of styrene monomer from polystyrene, the system comprising:
(d) a pyrolysis unit for pyrolyzing polystyrene to produce a pyrolysis gas containing C₅₋ light products, benzene, toluene, ethylbenzene, styrene monomer and C₉₊ aromatic compounds; and
(e) a condenser for removing at least part of the C₅₋ light products from the pyrolysis gas to produce a depolymerization oil containing benzene, toluene, ethylbenzene, styrene monomer and C₉₊ aromatic compounds; the depolymerization oil being supplied, without intermediate fractionation, to the apparatus of claim 12.
